(19) United States
(12) Patent Application Publication
(10) Pub. No.: US 2012/0285271 A1
(43) Pub. Date: Nov. 15, 2012

(54) SIMULATION ARRANGEMENT FOR A SMOKING ARTICLE

(75) Inventors: Balager Ademe, Winston-Salem, NC (US); John Larkin Nelson, Lewisville, NC (US)

(73) Assignee: R. J. Reynolds Tobacco Company

(21) Appl. No.: 13/105,425

(22) Filed: May 11, 2011

(51) Int. Cl. G01N 33/00

(52) U.S. Cl. 73/865.9

(57) ABSTRACT

An apparatus is provided for simulating smoking of a smokable rod member. A receiver is configured to receive one end of the smokable rod member. An analyzer is in communication with the receiver and is configured to receive smoke from the smokable rod member upon combustion of the other end thereof. An air source is in communication with the receiver and is configured to mix air with the smoke from the smokable rod member. A container device is in communication with the receiver and the air source, and is configured to receive a smoke and air mixture. The container device is further configured to have an adjustable volume to vary an amount of the smoke and air mixture received thereby, and to be in communication with the analyzer, with the container device also being configured to expel the smoke and air mixture therefrom to the analyzer for the smoke and air mixture to be analyzed thereby.

### BACKGROUND OF THE DISCLOSURE

1. Field of the Disclosure

Aspects of the present disclosure relate to smoking articles and, more particularly, to apparatuses and methods for simulating smoking of a smoking article and to analyze the smoke product produced thereby.

2. Description of Related Art

In determining the burn characteristics of smoking articles, one aspect involves analyzing the smoke produced thereby. One manner of accomplishing such an analysis may involve, for example, a simulation arrangement configured to simulate the smoking activity associated with the smoking article. Such a simulation arrangement may involve a suction source for pulling a particular amount or volume of air and smoke through the filter element of the smoking article, wherein at least a portion of that amount or volume is then directed to an analyzer device for analysis. However, such a simulation arrangement is often configured for a particular physiology in terms of the amount or volume of the smoke sample collected from the smoking article. That is, the captured smoke volume may often be of a fixed or predetermined magnitude. One shortcoming of such an arrangement may be that differences in physiology between smokers may not be adequately addressed. For example, not all smokers have the same lung capacity, and a constant volume/amount of a sample collected by existing simulation arrangements may not provide a precise analysis with respect to a diverse population.

As such, there exists a need for a more versatile and adaptable simulation arrangement for simulating the smoking activity associated with a smoking article.

### SUMMARY OF THE DISCLOSURE

The above and other needs are met by aspects of the present disclosure, wherein one aspect provides an apparatus for simulating smoking of a smokable rod member having opposed ends, comprising a receiver configured to receive one end of the smokable rod member, and an analyzer in communication with the receiver and configured to receive smoke from the smokable rod member upon combustion of the other end thereof. An air source is in communication with the receiver and is configured to mix air with the smoke from the smokable rod member. A container device is in communication with the receiver and the air source, and is configured to receive a smoke and air mixture. The container device is further configured to have an adjustable volume to vary an amount of the smoke and air mixture received thereby, and to be in communication with the analyzer, with the container device also being configured to expel the smoke and air mixture therefrom to the analyzer for the smoke and air mixture to be analyzed thereby.

Aspects of the present disclosure thus address the identified needs and provide other advantages as otherwise detailed herein.

### BRIEF DESCRIPTION OF THE SEVERAL VIEWS OF THE DRAWING(S)

Having thus described the disclosure in general terms, reference will now be made to the accompanying drawings, which are not necessarily drawn to scale, and wherein:

FIG. 1 schematically illustrates a general simulation arrangement for simulating smoking of a smoking article, according to particular aspects of the present disclosure; and

FIG. 2 schematically illustrates a threaded member and drive member arrangement for adjusting a volume of a container device in a simulation arrangement for simulating smoking of a smoking article, according to particular aspects of the present disclosure.

### DETAILED DESCRIPTION OF THE DISCLOSURE

The present disclosure now will be described more fully hereinafter with reference to the accompanying drawings, in which some, but not all aspects of the disclosure are shown. Indeed, the disclosure may be embodied in many different forms and should not be construed as being limited to the aspects set forth herein; rather, these aspects are provided so that this disclosure will satisfy applicable legal requirements. Like numbers refer to like elements throughout.

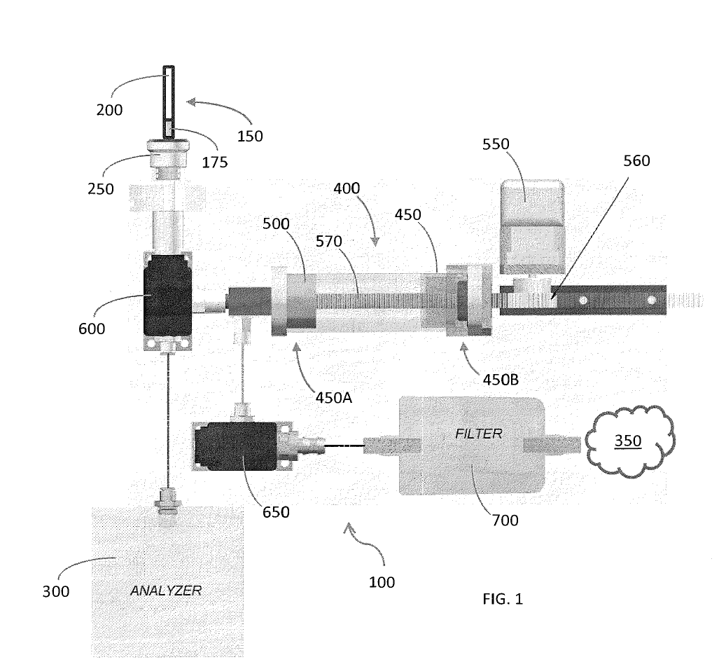

FIG. 1 schematically illustrates one aspect of an apparatus comprising a simulation arrangement for simulating the smoking or smoking activity associated with a smokable rod member, according to one aspect of the present disclosure, the apparatus being indicated by the numeral 100. The smokable rod member 150 may comprise any suitable smoking article such as, for example, a cigarette, cigar, or the like, as will be appreciated by one skilled in the art. Such a smokable rod member 150 is generally elongate and includes opposed ends 175, 200, wherein, in the case of a cigarette, one end 175 may comprise a filter element of the smokable rod member 150, while the opposed end 200 may comprise the tobacco rod or smokable end of the smokable rod member 150.

The apparatus 100 may comprise, for example, a single port smoking machine manufactured by Borgwaldt KC of Hamburg, Germany. As such the apparatus 100 may include a receiver or receiver device 250 configured to receive one end of the smokable rod member 150, such as, for example, the filter element end 175. In some instances, the receiver 250 may be configured to securely hold the smokable rod member 150 while forming a suitable engagement therewith, for example, similarly to the smokable rod member 150 being received by the mouth of the user.

An analyzer or analyzer device 300 is configured to be in communication with the receiver 250, and is configured to receive smoke from the smokable rod member 150 upon combustion or ignition of the smokable end 200 thereof. In particular aspects, the analyzer device 300 may be configured to analyze the composition of the smoke produced by the smokable rod member 150, including, for instance, the determination of particulate matter, chemical compounds, and gases contained therein.

An air source 350 may be in communication with the receiver device 250, wherein the air source 350 may be configured to mix air with the smoke from the smokable rod member 150. For example, air may be mixed with the smoke as the smoke is drawn from the smokable rod member 150 and/or when the smoke is being directed toward the analyzer device 300.

A container device 400 is in communication with the receiver device 250 and the air source 350, and is configured to receive the smoke from the smokable rod member 150 and/or a smoke and air mixture. The container device 400 may be further configured to have an adjustable volume so as to vary an amount of the smoke and/or smoke and air mixture received thereby. The container device 400 may also be configured to be in communication with the analyzer device 300. Accordingly, in some instances, the container device 400 may also be configured to expel the smoke and/or smoke and air mixture therefrom to the analyzer device 300 for the smoke and/or smoke and air mixture to be analyzed thereby.

In providing the adjustable volume aspect, the container device 400 may comprise a cylinder and piston arrangement. In such instances, the cylinder 450 may have opposed ends 450A, 450B, and the piston 500 may be configured to be axially movable along the cylinder 450 between the opposed ends 450A, 450B. The cylinder 450 may also be configured to be in communication with the receiver device 250, the air source 350, and the analyzer device 300, wherein the communication therewith may be realized about one end of the cylinder (i.e., about element 450A). As such, as the axially movable piston 500 moves from a position of minimum volume (about one end 450A of the cylinder 450) toward a position of maximum volume (about the other end 450B of the cylinder 450) of the cylinder 450, a suction is created, which serves to draw the smoke or smoke and air mixture from the receiver device 250 into the cylinder 450. Conversely, as the piston 500 reciprocates and moves back toward the one end 450A of the cylinder 450, the smoke or smoke and air mixture is expelled from the cylinder 450 to the analyzer device 300.

In some advantageous instances, the piston 500 may be configured to have an adjustable stroke with respect to the cylinder 450 for adjusting the volume of the container device 400. For example, the piston 500 may be operably engaged with a stepper actuator 550 via a gear and pinion arrangement engaged therebetween. In such instances, the stepper actuator 550 and/or the gear and pinion arrangement may be configured to be adjustable for adjusting the stroke of the piston 500, and thereby the maximum achievable volume of the container device 400. For example, a pitch and/or travel of one of the gear 560 and/or the pinion member 570 of the gear and pinion arrangement may be adjustable for adjusting the stroke of the piston 500. In another example, the stepper actuator 550 may be configured to have a digitally-controllable stroke which, in turn, controls the travel of the piston 500 and thereby controls the sampling volume associated with the container device 400. In such instances, the sampling volume of the container device 400 may essentially be controlled and adjusted in real time.

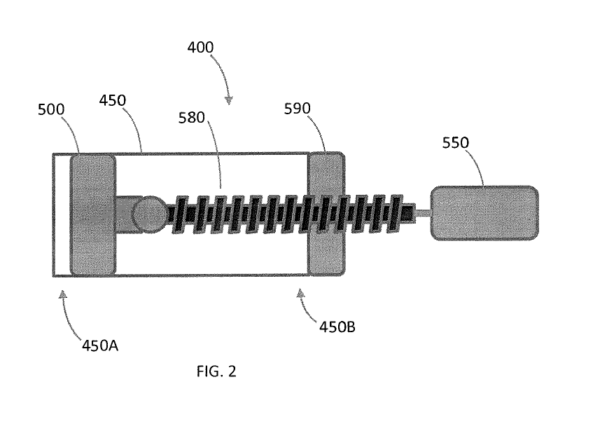

Of course, one skilled in the art will appreciate that the piston may be adjustably movable in various manners within the scope of the present disclosure. For example, the piston 500 may be operably engaged with the stepper actuator 550 via a threaded member 580 and drive member 590 arrangement engaged therebetween, wherein the stepper actuator 550 may be configured to be adjustable for adjusting the stroke of the piston 500, as shown, for example, in FIG. 2. Further, a pitch and/or travel of one of the threaded member 580 and/or drive member 590 of the threaded member and drive member arrangement may be adjustable for adjusting the stroke of the piston 500. In other examples, a lever arrangement may be operably engaged between the piston 500 and the stepper actuator 550. In still further examples, a pneumatic actuator or a hydraulic actuator may be operably engaged with the piston 500 for adjustably moving the piston 500 to thereby adjust the maximum achievable volume of the container device 400.

In order to appropriately direct the smoke or smoke and air mixture, a first valve member 600 may be disposed between and in communication with the receiver device 250, the analyzer device 300, and the container device 400. For example, such communication may be realized through appropriate piping, as will be appreciated by one skilled in the art. In some instances, the first valve member 600 may be configured to isolate the analyzer device 300 when the container device 400 is receiving the smoke or smoke and air mixture. For example, the first valve member 600 may be configured as a “three-way” valve having a branch in communication with each of the receiver device 250, the analyzer device 300, and the container device 400, wherein the first valve member 600 may be actuated to a “analyzer device closed” position prior to the piston 500 being actuated from the position of minimum volume toward the position of maximum volume (i.e., the analyzer device 300 is isolated prior the suction being initiated to draw the smoke from the smokable rod member 150 held by the receiver device 250). Further, after the maximum achievable volume (or otherwise the desired volume of the smoke or smoke and air mixture) is attained by the container device 400, the first valve member 600 may be actuated from the “analyzer device closed” position to a “receiver device closed” position, so as to isolate the receiver device 250 when the smoke or smoke and air mixture is being expelled from the container device 400 and toward the analyzer device 300. In some aspects of the present disclosure, the air source 350 may comprise atmosphere.

In other instances, as will be appreciated by one skilled in the art, the air source 350 may comprise oxygen or an oxygen-nitrogen mixture. In instances where the air source 350 comprises atmosphere, the apparatus 100 may further comprises a second valve member 650 disposed between and in communication with the first valve member 600 and the container device 400. For example, the second valve member 650 may be configured as a “three-way” valve having a branch in communication with each of the first valve member 600, the container device 400, and atmosphere, wherein the second valve member 650 may be actuated to an “atmosphere open” position prior to the piston 500 being actuated from the position of minimum volume toward the position of maximum volume (i.e., the second valve member 650 is opened to allow air into the apparatus 100 to mix with the smoke from the receiver device 250, prior the suction being initiated to draw the smoke and air mixture into the container device 400). Further, after the maximum achievable volume (or otherwise the desired volume of the smoke and air mixture) is attained by the container device 400, the second valve member 650 may be actuated from the “atmosphere open” position to an “atmosphere closed” position, so as to isolate the atmosphere when the smoke and air mixture is being expelled from the container device 400 and toward the analyzer device 300 (i.e., in conjunction with first valve member 600 being in the “receiver device closed” position.

One skilled in the art will appreciate, however, that the control of the air source 350 may be configured in different manners. For example, the branch of the second valve member 650 in communication with atmosphere may be configured in a single direction arrangement so as to allow atmosphere to be drawn therethrough and into the container device 400 when the container device 400 is receiving the smoke and air mixture, but will automatically be sealed (i.e. to prevent backflow to atmosphere) upon cessation of the suction from the container device).

In some aspects, in order to avoid compromising the analysis of the smoke and air mixture, a filter device 700 may be operably engaged with the second valve member 650 about the port thereof directed to atmosphere, wherein the filter device 700 is operatively disposed between the second valve member 650 and atmosphere. In such instances, the filter device may be configured to filter the air from atmosphere, prior to the air being drawn into the container device 400. In some further aspects, one of the second valve member 650 and the filter device 700 may be configured to be adjustable so as to be capable of selectively limiting the air drawn into the container device 400 when the container device 400 is receiving the smoke and air mixture. That is, in some instances, the filter device 700 and/or the second valve member 650 may be configured to be capable of selectively limiting the maximum amount of air which can be drawn into the container device 400.

Many modifications and other aspects of the disclosures set forth herein will come to mind to one skilled in the art to which these disclosures pertain having the benefit of the teachings presented in the foregoing descriptions and the associated drawings. For example, in addition to the adjustable volume of the container device 400 disclosed herein, the rate of change of the volume of the container device 400 may be configured to be adjustable or otherwise variable, for instance, to vary a constant rate of change or to alter a volume rate profile which may exhibit one or more transitions over the course of a single stroke of the piston 500 in the cylinder 450. In other instances, the air source 350 may be integrated into the apparatus 100 so as to be implemented to flush the container device 400, the analyzer device 300, and/or any tubing or components disposed in communication therewith between smoke sample collections for analysis. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

That which is claimed:

1. An apparatus for simulating smoking of a smokable rod member having opposed ends, comprising:
a receiver configured to receive one end of the smokable rod member;
an analyzer in communication with the receiver and configured to receive smoke from the smokable rod member upon combustion of the other end thereof;
an air source in communication with the receiver and configured to mix air with the smoke from the smokable rod member; and
a container device in communication with the receiver and the air source, and configured to receive a smoke and air mixture, the container device further being configured to have an adjustable volume to vary an amount of the smoke and air mixture received thereby, and to be in communication with the analyzer, the container device also being configured to expel the smoke and air mixture therefrom to the analyzer for the smoke and air mixture to be analyzed thereby.

2. An apparatus according to claim 1, wherein the container device comprises a cylinder and piston arrangement, with the cylinder having opposed ends and the piston being configured to be axially movable along the cylinder between the opposed ends, the cylinder being in communication with the receiver, the air source, and the analyzer about one end thereof, and the piston being axially movable away from the one end to draw the smoke and air mixture into the cylinder and toward the one end to expel the smoke and air mixture from the cylinder to the analyzer, the piston having an adjustable stroke for adjusting the volume of the container device.

3. An apparatus according to claim 2, wherein the piston is operably engaged with a stepper actuator via a gear and pinion arrangement engaged therebetween, the stepper actuator being configured to be adjustable for adjusting the stroke of the piston.

4. An apparatus according to claim 3, wherein a pitch of one of the gear and the pinion is adjustable for adjusting the stroke of the piston.

5. An apparatus according to claim 1, further comprising a first valve member disposed between and in communication with the receiver, the analyzer, and the container device, the first valve member being configured to isolate the analyzer when the container device is receiving the smoke and air mixture, and to isolate the receiver device when the smoke and air mixture is being expelled from the container device.

6. An apparatus according to claim 5, wherein the air source is atmosphere and the apparatus further comprises a second valve member disposed between and in communication with the first valve member and the container device, the second valve member being in communication with atmosphere and being configured in a single direction arrangement so as to allow air from atmosphere to be drawn therethrough and into the container device when the container device is receiving the smoke and air mixture.

7. An apparatus according to claim 6, further comprising a filter device operably engaged with the second valve member and disposed between the second valve member and atmosphere, the filter device being configured to filter the air from atmosphere prior to the air being drawn into the container device.

8. An apparatus according to claim 7, wherein one of the second valve member and the filter device is configured to be adjustable so as to be capable of selectively limiting the air drawn into the container device when the container device is receiving the smoke and air mixture.

9. An apparatus according to claim 2, wherein the piston is operably engaged with a stepper actuator via a threaded member and drive member arrangement engaged therebetween, the stepper actuator being configured to be adjustable for adjusting the stroke of the piston.

10. An apparatus according to claim 9, wherein a pitch of one of the threaded member and drive member is adjustable for adjusting the stroke of the piston.
